(19) United States
(12) Patent Application Publication
(10) Pub. No.: US 2012/0148015 A1
(43) Pub. Date: Jun. 14, 2012

(54) COMPUTER TOMOGRAPHY DEVICE

(75) Inventor: Martin Simon, Uhldingen-Muehlhofen (DE)

(73) Assignee: Wenzel Volumetrik GmbH, Singen (DE)

(21) Appl. No.: 13/389,318

(22) PCT Filed: Aug. 5, 2010
(86) PCT No.: PCT/EP2010/004802

§ 371 Date: Feb. 23, 2012

(30) Foreign Application Priority Data
Aug. 7, 2009 (DE) 10 2009 036 579.6

(51) Int. Cl. A61B 6/03; G01N 23/04

(52) U.S. Cl. 378/19; 378/20

(57) ABSTRACT

A computer tomography device for non-medical applications, in particular a non-medical material or workpiece test, having a sensor carrier unit comprising a plurality of individual pixels provided adjacent to one another, said sensor carrier unit being designed to detect invasive radiation of an x-ray radiation source by a detector surface. The detector surface extends in at least one plane in the shape of an arc, wherein the sensor carrier unit has a contour that is arced at least in sections and/or comprises a plurality of individual detector elements (20) arranged in a faceted shape, each comprising a flat detector surface (3), disposed adjacent to and/or adjoining one another along an arced line (6), and an object carrier, designed as a rotary plate (30), for a workpiece to be subjected to tomographic inspection is provided in a beam path between the x-ray radiation source (1) and the sensor carrier unit.

The present invention relates to a computer tomography device according to the introductory clause of the main claim.

In addition to the field of medical tomography, in recent times in particular x-ray tomography has also established itself for the testing of workpieces or materials, wherein, in an analogous manner to human or veterinary computer tomography, workpieces are acted on and transilluminated by a high power x-ray beam as invasive radiation. The workpiece, as the measurement object, is situated here between a (high power) x-ray source and an electronic x-ray detector, which converts the received ray signal in a suitable manner into a signal which is able to be evaluated electronically, and then by means of otherwise known methods of digital imaging constructs from this signal the desired image, including the image of any defects, cavities or suchlike of a workpiece.

In addition to the construction of the x-ray source, in particular the realization of the x-ray detector is critical for the image quality, resolution and an image noise of object images which are to be acquired in such a way. In an otherwise known manner, this is realized as an array of a plurality of detector pixels arranged in a planar manner, which for detecting the x-ray radiation and for a subsequent conversion into semiconductor-based detectable radiation are provided with scintillator coatings.

From the prior art, it is known here to select the detector size (array size) in accordance with the dimensions of a measurement object or respectively following the geometry in a tomograph interior. A disadvantage due to the principle already exists here in that an x-ray source, to be assumed in an idealized manner as punctiform, which radiates onto a flat detector surface, brings a different radiation intensity (conditional upon distance) and onto the individual pixels of the detector, irrespective of whether a respective individual pixel is arranged rather more centrally or at the edge. Furthermore, the non-vertical impingement of the rays onto a respective pixel leads to a further reduction of the intensity.

A consequence of this is an impairment of the detector dynamics or respectively contrast resolution, and non-homogeneous solid angles between the x-ray exit point and the respective pixels, having a disadvantageous effect on a realizable image resolution and image quality.

A further disadvantage of detector devices known from the prior art lies in that over the duration of use the strongly ionizing x-ray radiation leads to a degradation of the detector pixels—and detector electronics, in other words, the usable running time of conventional detectors is limited.

Finally, it is to be presupposed as being known from the prior art, in the case of a required detector surface having a particularly large area, to compose the latter from a plurality of individual detectors (respectively having a flat detector surface). However, the problem exists here that at a transition between two adjacent individual detectors pixel inaccuracies are produced in that practically no transition-free joining together of individual detectors which are adjacent to each other is possible—this lies in that the electronic components extend beyond the respective edge lengths of the (pure) detector surface and thus an arrangement adjacent to each other must then always also take maximum edge lengths into consideration.

In view of this initial situation from the prior art, it is therefore an object of the present invention to provide an improved x-ray detector device in which the imaging quality and resolution of a detected x-ray signal, in particular of a punctiform x-ray source, is improved and in particular differences in quality in the imaging between individual pixels at the edge and individual pixels in the centre are reduced. Furthermore, it is an object of the present invention to prevent disadvantageous impairment or degradation of electronic image acquisition arrays by the invasive, ionizing radiation or respectively to minimize the disadvantageous effect thereof on the detector electronics. Finally, it is an object of the present invention to provide the preconditions for a pixel-accurate arrangement of individual detectors which are adjacent to each other, without an effective detector surface being expanded or distorted by non-detecting housing components.

The problem is solved by the computer tomography device with the features of the main claim and by the device with the features of the independent claim 9; advantageous further developments of the invention are described in the subclaims.

The computer tomography device according to the invention makes provision that a sensor carrier unit designed as a detector device stands opposite an (otherwise known) x-ray radiation source, wherein an object carrier, designed as a rotary plate, extends, at least in the operating state of the device, into the beam path between the x-ray radiation source and the detector device, wherein workpieces or respectively materials to be subjected to inspection in a suitable manner are then provided on the rotary plate. It is preferred here within the scope of the invention to design a rotation axis of the rotary plate so that it is aligned perpendicularly to the first plane, namely the plane of the arced detector surface. Hereby, a support surface of the rotary plate, assumed for instance as being flat, then lies in the first plane or parallel thereto, wherein in this way advantageously and according to a further development conveniently also large objects can be detected tomographically using the advantages, to be explained in further detail below, which are realized through the arc form of the detector surface.

In an advantageous manner according to the invention, the detector surface according to the present invention extends at least in a plane, namely the first plane, in the shape of an arc, wherein “in the shape of an arc” within the scope of the invention means not only a continuous, kink-free arc shape, but in particular also a facet-like series of flat individual surfaces, following an arc shape, which according to a preferred embodiment are arranged along an arc shape.

According to this first aspect of the solution, the geometric preconditions are thereby provided to reduce or respectively rule out differences in image quality and sharpness of a punctiform x-ray source through the detector device, because, particularly when the arc shape has an (approximately) constant radius in relation to the mid-point of the punctiform x-ray source, approximately each individual pixel is spaced equally along the arc, therefore the best preconditions exist for an optimum image quality (wherein in the embodiment according to the invention in facet form, the respective image faults remain able to be controlled and mastered by suitable configuration of the individual facets and the number of individual detectors).

Also, the provision, angled in the manner of facets, of individual detectors adjoining one another on their detector surface, already enables in a very far-reaching way the realization of a transition, uninterrupted with regard to pixels, of the detector surface between adjacent individual sensors, so that also in this way geometric advantages are realized (through the arrangement angled with respect to each other in the first plane, namely in particular housing dimensions of an individual detector which are widened on the rear side remain without an influence on the overall arrangement on the detector surface).

In a preferred further development of this solution, in addition provision is made according to the invention to geometrically separate the actual (front side) detector surface as radiation entry surface from a (typically) semiconductor-based, for instance CCD detector array, wherein radiation- or light-conducting means are provided between these. This provision likewise has a positive effect in two respects with regard to the problem which is posed: On the one hand, it enables the radiation- or respectively light-conducting functionality to displace towards the rear the (potentially) voluminous, wide electronics components relative to the detector surface, and therefore to prevent these from opposing a pixel-accurate transition between individual detectors which are adjacent to each other. On the other hand, it even enables the configuration of the radiation- or respectively light-conducting means provided according to a further development, by means of an entry and exit surface, offset relative to each other, for the radiation which is to be transmitted, to place the (sensitive) detector elements entirely out from the (x-ray) beam path, so that the disadvantageous degradation of semiconductor-based sensor arrays by invasive radiation can be efficiently prevented or reduced. In fact this second provision according to the invention is particularly assisted by apertures provided according to a further development, for instance in the form of a collimator which, suitably placed in front of the detector surface, makes provision that incident x-ray radiation is concentrated only onto the detector surface.

In the preferred further development, it is advantageous here on the one hand to configure the radiation- or respectively light-conducting means, which can further preferably be realized as a light-conducting fibre plate or suchlike light-conducting body, so that in the manner of a parallelogram the entry and exit surfaces enable an offsetting of the beam path. Alternatively, provision is made within the scope of a preferred embodiment, to connect the entry and exit surfaces via an arc shape and in this respect to arrange them at an angle relative to each other (which can typically be even 90°, i.e. the radiation- or respectively light-conducting means angle a typically horizontally incident x-ray beam, if applicable after corresponding modification, through 90° downwards in the manner of an arc). Through suitable protective or respectively facing measures, it is then possible to protect a detector which is arranged there, namely at the exit surface, directly from the harmful invasive radiation.

Whilst it is advantageous according to the present invention for the approximation of as ideal an arc shape as possible to arrange as many individual facets as possible (with respectively flat detector surface) adjacent to each other, within the scope of the invention a minimum segment number of 3, further preferably of 5, has proved to be advantageous.

Independent protection within the scope of the invention is claimed for an x-ray detector device in which—in the direction of the beam path—a second detector surface can be provided, either adjacent or overlapping, in front of or next to the first detector surface (to be provided so as to be stationary). This second detector surface, typically on a second carrier unit (but alternatively also on the same carrier unit), can be brought, swiveled, shifted or placed by suitable mechanical or electromechanical provisions before or respectively onto the provided position (alternatively can be securely present in the same plane adjacent to the first detector surface), wherein in this way in a very simple and elegant manner and without great constructional/geometric modifications in a tomograph interior an adaption can take place to particular detection requirements of an alternative measurement object, or respectively various measurement processes can be carried out in succession.

Further advantages, features and details of the invention will emerge from the following description of preferred example embodiments and with the aid of the drawings; these show in:

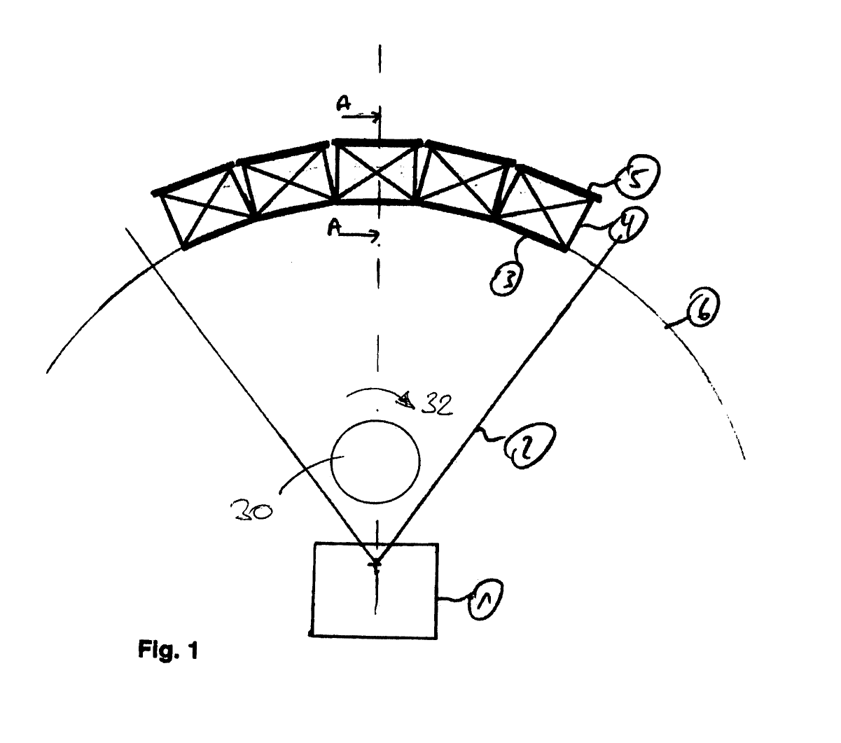

FIG. 1: a diagrammatic top view onto the computer tomography device according to a first embodiment of the invention with five individual detectors arranged in the manner of facets along an arc shape, relative to an x-ray source;

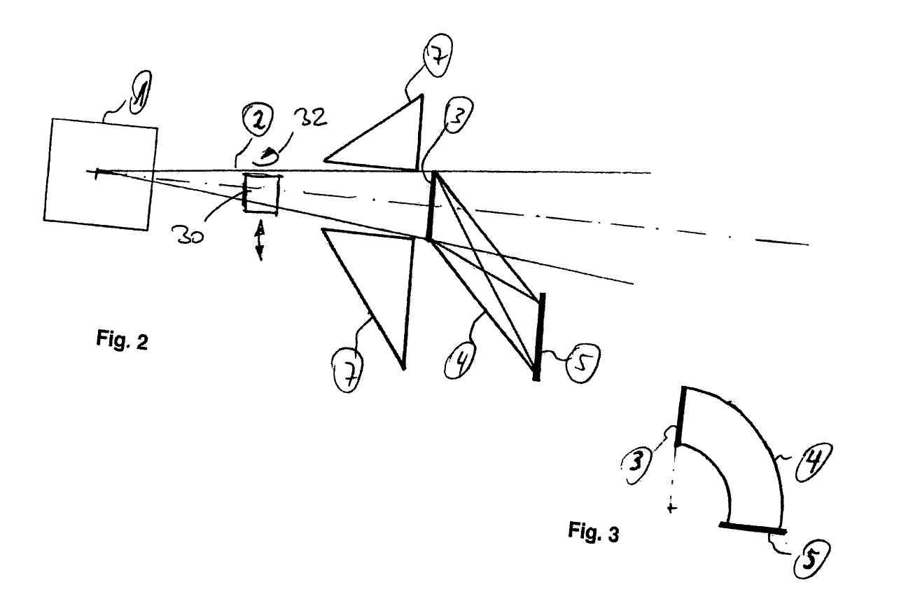

FIG. 2: a diagrammatic sectional view analogous to the section line A-A in FIG. 1, wherein in addition a collimator arrangement is shown for beam concentration or respectively for producing a beam aperture and in addition the exit surface is arranged offset downwards;

FIG. 3: an alternative construction of the radiation- or light-conducting means of the detector device with respect to the illustration of FIG. 2;

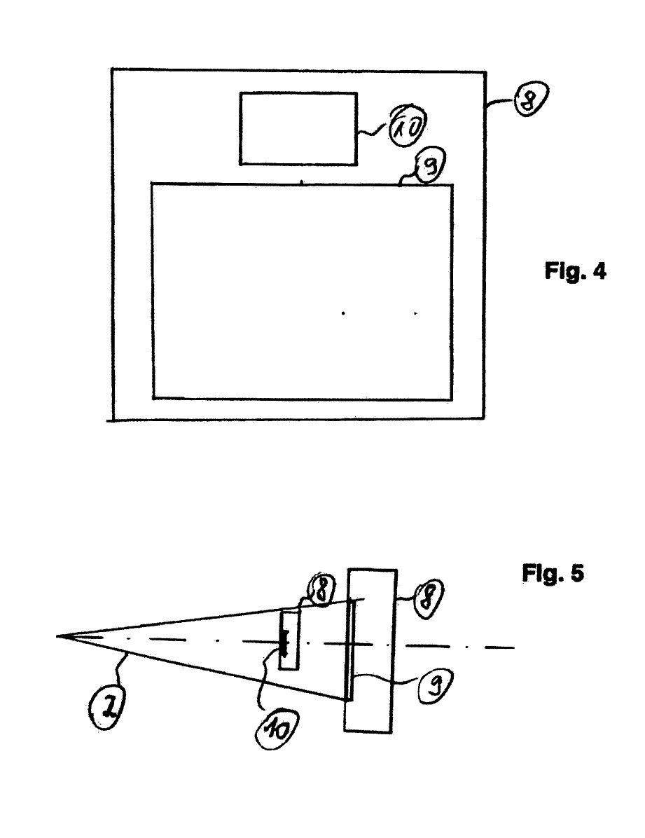

FIG. 4: a diagram view of a detector device with a first and a second detector surface, which are provided adjacent to each other, and

FIG. 5: a diagrammatic longitudinal section arrangement, with a first and a second detector surface, wherein a second carrier unit with the second detector surface is able to be brought movably into the beam path in front of the first detector surface.

FIG. 1 illustrates in the diagrammatic top view a first embodiment of the present invention: A plurality of five individual detectors 20, as can be seen in the top view of FIG. 1, is arranged in the horizontal plane along the circular arc 6 (radius 2), so that within the spanned angle 2 a facet shape is produced in relation to an x-ray source 1 (to be assumed as punctiform). An object carrier, formed as rotary plate 30, is placed in the beam path, ideally along the line A-A, wherein x-ray radiation from the source 1 then penetrates an object held thereon and is received by the detector arrangement 20.

The rotary plate has, accordingly, a rotation axis which runs perpendicularly to the plane of the drawing of FIG. 1, so that a rotation direction along the base arrow 32 of the rotary plate takes place in accordance with the arc-shaped curvature along the circular arc 6.

In addition, in accordance with a further development, provision is also made within the scope of preferred embodiments of the invention, to configure the rotary plate 30 so as to be movable vertically along the rotation axis, e.g. by drive means provided accordingly on or respectively in the rotary plate. In this way then e.g. a large (high) workpiece, which exceeds the detector height in projection, can be scanned successively.

It is first of all evident that this arrangement in the form of a circular arc has considerable geometric advantages over an individual flat detector with regard to a consistent length of the beam path: Only with the (drastically reduced) flatness faults of the individual segments, differences in length still occur between the punctiform x-ray source 1 and a respective detector surface 3.

As FIGS. 2 and 3 show additionally, here each of the individual detector elements 20 is constructed as a sequence, offset spatially along the beam path, from a detector surface 3 (here through otherwise known coatings a scintillator surface is applied, by which the x-ray photons are converted into detectable photons), wherein the entry surface which is thus formed is connected via a light-conducting zone 4 (realized in the illustrated example embodiment of FIG. 2 as a parallelogram-like light-conducting fibre plate) with an exit surface 5, provided offset relative to the entry surface, wherein here then in a manner not shown in further detail a detector array is provided for the generating of corresponding electronic signals. FIG. 2 additionally illustrates that through the parallelogram shape of the light conductor 4 which is shown, the light exit surface (and accordingly the detector array which is able to be provided there) lies outside the beam path 2 of the x-ray radiation, consequently is therefore no longer impaired by this invasive radiation.

FIG. 2 additionally illustrates by reference number 7 a collimator arrangement, by which the radiation incidence of the x-ray beam 2 can be limited only to the entry surface 2 (detector surface).

FIG. 3 illustrates an alternative form of realization of the radiation- or respectively light conductor 4. In the example of FIG. 3, this conductor (again suitably realized as a light conductor fibre arrangement or suchlike in the manner of a body) is configured in an arc shape, so that between the entry surface 3 and the exit surface 5 in the example a 90° angle is able to be realized. Here, also, the advantage of an arrangement of optically downstream detector arrays outside the x-ray beam path can be realized.

FIGS. 4 and 5 illustrate an alternative form of solution of the present invention. Thus, the frontal view of FIG. 4 shows how within a detector arrangement 8 two individual detector surfaces 9 (having a large surface) and 10 (having a small surface), adjacent to each other here and not overlapping, can be provided. This arrangement advantageously makes it possible, particularly in connection with a vertically adjustable workpiece carrier, to use different object sizes, measurement methods or suchlike without major modification steps or suchlike geometric alterations in a tomograph interior, simply by different connection of the detectors 9 or respectively 10 and suitable positioning of a workpiece relative to these detectors and the radiation source.

FIG. 5 illustrates a further variant of such an arrangement with two detector surfaces, wherein these overlap each other here in the direction of the beam path 2 and a second (smaller) detector surface 10 can be moved by carrier means, only indicated diagrammatically, in a movable manner (e.g. able to be pivoted, to be set or suchlike) in front of the stationary carrier unit 8 with the stationary detector 9. Such a procedure also enables in a simple and elegant manner an adaptation to different radiation, object and measurement conditions.

The present invention is not restricted to the example embodiments which are shown. Thus, on the one hand, it lies within the scope of the invention to configure the detector units 4, in particular by means of the light conducting arrangements which are provided, in a suitable manner for a respective case of application wherein these, as shown in FIGS. 2 and 3, can be displaced or respectively bent, but alternatively also can simply bridge a linear optical distance. It also lies within the scope of the invention to provide an arbitrary plurality of individual sensors according to FIG. 1 to form an arc shape, wherein this arc shape is in fact advantageously in the shape of a circular arc, but the arc shape is not, however, limited to the circular arc shape. It is also possible within the scope of preferred further developments to provide an arc shape two-dimensionally (i.e. in calotte shape), so that an arc shape could also be realized in the vertical, in relation to the plane of the figure of FIG. 2.

1. A computer tomography device for non-medical applications, comprising:
a sensor carrier unit comprising a plurality of individual pixels provided adjacent to one another, said sensor carrier unit being designed to detect invasive radiation of an x-ray radiation source by a detector surface, the detector surface extends in at least a first plane in the shape of an arc, wherein the sensor carrier unit has a contour that is arced at least in sections and/or comprises a plurality of individual detector elements (20) arranged in a faceted shape, each comprising a flat detector surface (3), disposed adjacent to and/or adjoining one another along an arced line (6), and an object carrier, designed as a rotary plate (30), for a workpiece to be subjected to tomographic inspection is provided in a beam path between the x-ray radiation source (1) and the sensor carrier unit.

2. The device according to claim 1, wherein the detector surface as radiation entry surface is connected via radiation- and/or light-conducting means (4) with at least one semiconductor-based detector array for electronic signal generation.

3. The device according to claim 1, wherein the detector surface (3) has scintillator means which are formed for converting x-ray photons into photons which are detectable by a semiconductor-based detector array.

4. The device according to claim 2, wherein the radiation- or respectively light-conducting means are formed as an arrangement of a plurality of light-conducting fibres running parallel to each other and/or as a light-conducting fibre plate (4).

5. The device according to claim 2, wherein the radiation- and/or light-conducting means are formed in longitudinal section such that a flat entry surface (3) of the radiation- or respectively light-conducting means is offset relative to a flat exit surface (5) of the radiation- or respectively light-conducting means, in particular is offset in the manner of a parallelogram or in an angular and/or arc shape.

6. The device according to claim 5, wherein an offsetting between the entry surface and the exit surface is arranged so that a radiation entering along an x-ray beam path into the entry surface emerges from the exit surface such that a detector array provided thereon lies outside the beam path.

7. The device according to claim 1, wherein collimator means (7) are associated with the detector surface such that these act for suppression of invasive radiation outside the detector surface.

8. The device according to claim 1, wherein the arc shape of the detector surface is a circular path (6) on which in the manner of facets at least three of the individual detectors (20) are arranged adjoining each other.

9. A computer tomography device for non-medical applications, comprising a sensor carrier unit (8) comprising a plurality of individual pixels provided adjacent to one another, which are designed to detect invasive radiation by a detector surface (9, 10), the detector surface has a first stationary detector surface (9) and a second detector surface (10), which, in relation to a beam path of the invasive radiation, can be arranged adjacent to the first detector surface on the sensor carrier unit or removably and/or movably in front of the first detector surface.

10. The device according to claim 9, wherein the second detector surface (10) has a reduced detector surface with respect to the first stationary detector surface (9).

11. The device according to claim 10, wherein the second detector surface is formed onto the stationary detector surface and/or the sensor carrier unit by swiveling, displacing, detachable setting on, screwing on or placing on of a second carrier unit.
